SEQUENCE LISTING (26 sequences)

SEQ ID NO: 1 - DNA, 27 bp, Artificial
  Oligonucleotide primer with homology to Human p53 DNA sequence
gagggatccgaggagccgcagtcagat

SEQ ID NO: 2 - DNA, 26 bp, Artificial
  Oligonucleotide primer with homology to Human p53 DNA sequence
gagctcgagtcagtctgagtcaggcc

SEQ ID NO: 3 - DNA, 27 bp, Artificial
  Oligonucleotide primer with homology to human K-Ras DNA sequence
gagggatccactgaatataaacttgtg

SEQ ID NO: 4 - DNA, 27 bp, Artificial
  Oligonucleotide primer with homology to human K-Ras DNA sequence
gagctcgagttacataattacacactt

SEQ ID NO: 5 - DNA, 27 bp, Artificial
  Oligonucleotide primer with homology to human Smad4 DNA sequence
gagggatccgacaatatgtctattacg

SEQ ID NO: 6 - DNA, 27 bp, Artificial
  Oligonucleotide primer with homology to human Smad4 DNA sequence
gagctcgagtcagtctaaaggttgtgg

SEQ ID NO: 7 - DNA, 27 bp, Artificial
  oligonucleotide primer with homology to Human Beta-catenin DNA sequence
gagggatccgctactcaagctgatttg

SEQ ID NO: 8 - DNA, 30 bp, Artificial
  oligonucleotide primer with homology to Human Beta-catenin DNA sequence
gaggtcgactcaaccagctaaacgcactgc

SEQ ID NO: 9 - DNA, 27 bp, Artificial
  oligonucleotide primer with homology to Human c-myc DNA sequence
gagggatccctcaacgttagcttcacc

SEQ ID NO: 10 - DNA, 27 bp, Artificial
  oligonucleotide primer with homology to Human c-Myc DNA sequence
gagctcgagttacgcacaagagttccg

SEQ ID NO: 11 - DNA, 55 bp, Artificial
  homologous  to mammalian expression vector
atcagccgccaccatggactacaaggacgacgatgacaagggatctacttcgaaa

SEQ ID NO: 12 - DNA, 54 bp, Artificial
  homologous to mammalian expression vector
aaaaatgaacaaggatccgaattcaaaaagcttctcgagagtacttctagagcg

SEQ ID NO: 13 - DNA, 27 bp, Artificial
  oligonucleotide primer with homology to HIV nucleic acid sequence
gagggatcccctatagtgcagaacatc

SEQ ID NO: 14 - DNA, 30 bp, Artificial
  oligonucleotide primer with homology to HIV nucleic acid sequence
gagctcgagtcacaaaactcttgccttatg

SEQ ID NO: 15 - DNA, 27 bp, Artificial
  oligonucleotide primer with homology to Pneuomocystis jiroveci DNA sequence
gagggatccactgaatataaacttgtg

SEQ ID NO: 16 - DNA, 27 bp, Artificial
  oligonucleotide primer with homology to Pneuomocystis jiroveci DNA sequence
gagctcgagttacataattacacactt

SEQ ID NO: 17 - DNA, 23 bp, Artificial
  oligonucleotide primer with homology to Pneuomocystis jiroveci DNA sequence
gagggatccgatttcgatccaac

SEQ ID NO: 18 - DNA, 30 bp, Artificial
  oligonucleotide primer with homology to Pneuomocystis jiroveci DNA sequence
gagctcgagctaaatcatgaacgaaataac

SEQ ID NO: 19 - DNA, 29 bp, Artificial
  oligonucleotide primer with homology to Pneuomocystis jiroveci DNA sequence
gagggatcctgtaataaatggtctagaag

SEQ ID NO: 20 - DNA, 28 bp, Artificial
  oligonucleotide primer with homology to Pneuomocystis jiroveci DNA sequence
gagctcgagctacccgctcaccctcaag

SEQ ID NO: 21 - DNA, 29 bp, Artificial
  oligonucleotide primer with homology to HBV nucleic acid sequence
gagggatccagacattgacccttataaag

SEQ ID NO: 22 - DNA, 26 bp, Artificial
  oligonucleotide primer with homology to HBV nucleic acid sequence
gagctcgagctaacattgagattccg

SEQ ID NO: 23 - DNA, 27 bp, Artificial
  oligonucleotide primer with homology to HCV nucleic acid sequence
gagagatcttccggttcctggctaagg

SEQ ID NO: 24 - DNA, 30 bp, Artificial
  oligonucleotide primer with homology to HCV nucleic acid sequence
gagctcgagtcagcagcacacgacatcttc

SEQ ID NO: 25 - DNA, 27 bp, Artificial
  oligonucleotide primer with homology to HCV nucleic acid sequence
gagggatccgcggtggactttatccct

SEQ ID NO: 26 - DNA, 31 bp, Artificial
  oligonucleotide primer with homology to HCV nucleic acid sequence
gagtctagatcaacagctgttgcagtctatc

(19) United States
(12) Patent Application Publication
(10) Pub. No.: US 2007/0259336 A1
(43) Pub. Date: Nov. 8, 2007

(54) Methods for the detection of analytes in a sample

(75) Inventors: Peter Burbelo, Washington, DC (US); Thomas L. Mattson, Germantown, MD (US)

Correspondence Address: ROPES & GRAY LLP;PATENT DOCKETING 39/41, ONE INTERNATIONAL PLACE, BOSTON, MA, 02110-2624, US

(73) Assignee: Georgetown University, Washington, DC (US)

(21) Appl. No.: 11/318,134

(22) Filed: Dec. 23, 2005

Related U.S. Application Data
(60) Provisional application No. 60/638,811, filed on Dec. 23, 2004.

(51) Int. Cl. C12Q 1/70; G01N 33/53; G01N 33/566

(52) U.S. Cl. 435/5; 435/7.21; 435/7.23; 435/7.9; 436/501

(57) ABSTRACT

A method for detecting the presence or concentration of an analyte in a sample, wherein: a) the sample is contacted with an immobilized first binding reagent which is capable of binding the analyte if present in the sample; b) the sample is contacted with a second binding reagent which comprises a fusion protein having a reporter domain and a binding domain, and which is capable of binding the analyte if present in the sample, the first and second binding reagents being capable of binding the analyte simultaneously if present in the sample, such that said second binding reagent becomes immobilized through the analyte bound to the first binding reagent; and detecting whether the second binding reagent has become immobilized to thereby detect the presence or concentration of said analyte.

### CROSS REFERENCE TO RELATED APPLICATIONS

This application claims the benefit of U.S. Provisional Application No. 60/638,811, filed Dec. 23, 2004. The entire teachings of the referenced application are incorporated herein by reference.

### BACKGROUND OF THE INVENTION

1. Field of the Invention

The present invention generally relates to methods for detecting the presence or concentration of an analyte in a sample utilizing an immobilized first binding reagent and a second binding reagent which comprises a fusion protein having a reporter domain and a binding domain. The methods may be used to detect rapidly and with high sensitivity, the presence or progress of, e.g., infectious diseases, inflammatory diseases, autoimmune diseases and cancer.

2. Description of the Related Art

Assays detecting human antigen-specific antibodies are medically useful. However, the usefulness of existing simple immunoassay formats is limited by technical considerations such as sera antibodies to contaminants in insufficiently pure antigen, a problem likely exacerbated when antigen panels are screened to obtain clinically useful data.

Most immunoassays use bacterial-expressed proteins for detecting antigen-specific antibodies in human sera [2]. However, since such antigens do not carry post-translational modifications or may fold incorrectly, some immunoassays employ antigens produced in either yeast or insect cells. While these antigens may fold correctly and carry post-translational modifications, they will not carry either mammalian- or disease-specific posttranslational modifications. Tests employing bacterial-produced proteins can produce high backgrounds because it is difficult to completely eliminate or block serum antibodies reactive with trace amounts of bacterial contaminants present in most antigen preparations, even in pharmaceutical grade preparations [3]. The present invention overcomes these problems.

### BRIEF SUMMARY OF THE INVENTION

In one aspect, the present invention provides a method for detecting the presence or concentration of an analyte in a sample, said method comprising:
a) contacting said sample with an immobilized first binding reagent, said reagent capable of binding the analyte if present in the sample;
b) contacting said sample with a second binding reagent which comprises a fusion protein having a reporter domain and a binding domain, said binding domain being capable of binding the analyte if present in the sample, and said first and second binding reagents being capable of binding the analyte simultaneously if present in the sample, such that said second binding reagent becomes immobilized through the analyte bound to the first binding reagent; and
c) detecting whether the second binding reagent has become immobilized to thereby detect the presence or concentration of said analyte.

In another aspect, the present invention provides a method for monitoring the course of a disease in a patient having need of such monitoring, said method comprising:
a) contacting a first fluid sample from said patient with an immobilized first binding reagent, said reagent capable of binding to an analyte in the sample whose level is indicative of the progress of the disease;
b) contacting said sample with a second binding reagent which comprises a fusion protein having a reporter domain and a binding domain, said binding domain being capable of binding the analyte if present in the sample, and said first and second binding reagents being capable of binding the analyte simultaneously, such that said second binding reagent becomes immobilized through the first binding reagent;
c) detecting the extent to which the second binding reagent has become immobilized to thereby quantify the concentration of said analyte; and
d) repeating steps (a)-(c) on a second fluid sample collected from said patient at a time subsequent to the collection of the first fluid sample to thereby monitor the concentration of the analyte over time.

In yet another aspect, the present invention provides a method for detecting the presence or concentration of an analyte in a sample, said method comprising, in any order:
a) contacting said sample with a first binding reagent, said reagent capable of binding the analyte if present in the sample;
b) contacting said sample with a second binding reagent which comprises a fusion protein having a reporter domain and a binding domain, said binding domain being capable of binding the analyte if present in the sample, and said first and second binding reagents being capable of binding the analyte simultaneously if present in the sample;
c) immobilizing said first binding reagent such that when the first and second binding reagents are bound to the analyte, said second binding reagent becomes immobilized through the analyte bound to the first binding reagent; and
d) detecting whether the second binding reagent has become immobilized to thereby detect the presence or concentration of said analyte.

In yet another aspect, the present invention provides a kit for detecting the presence or concentration of an analyte in a sample, said kit comprising:
a) an immobilized first binding reagent, said reagent capable of binding the analyte if present in the sample;
b) a second binding reagent which comprises a fusion protein having a reporter domain and a binding domain, said binding domain being capable of binding the analyte if present in the sample, and said first and second binding reagents being capable of binding the analyte simultaneously if present in the sample, such that said second binding reagent becomes immobilized through the first binding reagent; and
c) suitable packaging material.

### BRIEF DESCRIPTION OF THE DRAWINGS

FIG. 1 shows the results of immunoprecipitation experiments with commercial antibodies. Various amounts of anti-FLAG monoclonal, anti-p53 polyclonal or control (anti-WASP) polyclonal antibodies were mixed with 5 μl of a Cos1 extract containing Ruc-p53 for 1 h in the presence of protein A/G beads, processed and light units measured. The data shown is from one of three independent experiments giving similar results.

FIG. 2 shows the results of an immunoprecipitation assay with Ruc-p53 and a clinical serum sample. Graph A shows that the immunoprecipitation activity is proportional to incubation time. Tubes containing identical amounts of Ruc-p53 fusion protein extract (5 μl), patient 34 sera (1 μl) and protein A/G beads were incubated for 5, 30, 60, 90 and 120 min and processed for luciferase activity. Graph B shows the immunoprecipitation activity with various amounts of total crude patient 34 sera. Different amounts of patient sera (0.002 to 2 μl) were mixed with 5 μl of the Ruc-p53 fusion protein extract and incubated for 1 hour in the presence of protein A/G beads, processed and light units measured. The data shown is from one of three independent experiments giving similar results.

FIG. 3 shows the results of competition assays blocking Ruc-p53 immunoprecipitation using bacterially-produced antigen. Different amounts of E. coli-produced MBP-p53 were incubated with patient sera 34 (0.5 μl) or commercial anti-p53 antibody (25 ng) for 1 h. Protein A/G beads and Ruc-p53 extract were then added and incubated for an additional 1 h, processed and light units measured. The data shown is from one of two independent experiments giving similar results.

FIG. 4 depicts the structure of the pREN2 mammalian expression vector. Features indicated are CMV (cytomegalovirus) promoter, the N-terminal FLAG epitope and Ruc. Sequences for Ruc are in bold. cDNAs for tumor antigens were cloned downstream of Ruc between the BamHI-XhoI sites.

FIG. 5 depicts an assay within the scope of the present invention.

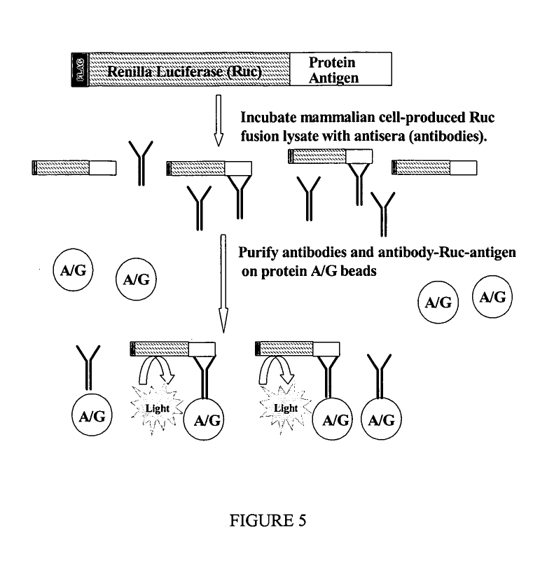

FIG. 6 presents data obtained from an experiment described in Example 2.

### DETAILED DESCRIPTION OF THE INVENTION

The present invention provides a rapid, simple and highly sensitive assay to detect the presence or quantify the concentration of an analyte in a sample. Many analytes are within the scope of the present invention. For example, the present assays may be used to detect the presence or quantify the concentration of analytes of medical or biochemical interest, such as antibodies, proteins, antigens, carbohydrates, lipids, etc. The sample containing the analyte to be detected may be any body fluid where the analyte may be found, including but not limited to blood, saliva, ascites, urine, cerebrospinal fluid, amniotic fluid, sputum and gastric fluid.

The first binding reagent of the present invention should be capable of binding to the analyte of interest if present in the sample. As used herein, the term “binding” is intended to mean any interaction or association between the first binding reagent and the analyte that will ultimately permit the analyte to be immobilized via the immobilized first binding reagent, and ultimately to immobilize the second binding reagent. Preferably the binding interaction will have a Kd of about 10⁻⁶, more preferably about 10⁻⁷, even more preferably about 10⁻⁸, and as high as about 10⁻¹⁴. In a preferred embodiment, such binding will be in the nature of a protein/protein or antigen/antibody interaction.

The first binding reagent should also be immobilized. Many immobilization schemes are well known to one of skill in the art, and include covalent immobilization on a solid support such as plastics, magnetic beads, nylon, carbohydrate-based supports, etc. The first binding reagent may be immobilized at any time during the process. For example, it may be immobilized before contact with the fluid sample suspected of containing the analyte. In another embodiment, the first binding reagent may be immobilized after it is contacted with the analyte and/or the second binding reagent. In the latter embodiment, the binding reaction(s) is carried out in solution, then the first binding reagent is subsequently (or simultaneously) immobilized by methods well-known to one of ordinary skill. For example, the first binding reagent in solution may be contacted with a solid medium having an affinity for the first binding reagent, for example a bead coated with an antibody that binds to the first binding reagent.

The first binding reagent may comprise any moiety that is capable of binding to the analyte. Preferred moieties include proteins and antibodies. When the analyte is an antibody, the first binding reagent preferably comprises a protein known to bind to the class of such antibody, such as protein A or protein G.

The second binding reagent of the present invention comprises a fusion protein having a reporter domain and a binding domain. The fusion protein may be made by conventional cloning techniques. The fusion protein may be expressed in a wide range of cells, including mammalian, yeast and plant cells. In a preferred embodiment, the fusion protein is expressed in mammalian cells or cell extracts, such as Cos cells, HeLa, Vero, CHO, NIH 3T3, 293, etc. The use of mammalian cells is particularly preferred for the following reasons. Most immunoassays use bacterial-expressed proteins for detecting antigen-specific antibodies in human sera [2]. However, since such antigens do not carry post-translational modifications or may fold incorrectly, some immunoassays employ antigens produced in either yeast or insect cells. While these antigens may fold correctly and carry post-translational modifications, they will not carry either mammalian- or disease-specific posttranslational modifications. Tests employing bacterial-expressed proteins can produce high backgrounds because it is difficult to completely eliminate or block serum antibodies reactive with trace amounts of bacterial contaminants present in most antigen preparations, even in pharmaceutical grade preparations [3]. Therefore, the use of mammalian-produced fusions can overcome those problems. In a preferred embodiment, such fusions will contain a post-translational modification, such as glycosylation, acetylation, lipidation (e.g., palmitoylation), phosphorylation, citrullination, etc.).

The binding domain of the second binding reagent is capable of binding the analyte if present in the sample, with “binding” being used in the same sense as above. Further, the first and second binding reagents should be capable of binding the analyte simultaneously if present in the sample. When that occurs, it will be apparent that the second binding reagent becomes immobilized through the analyte and the first binding reagent. The binding domain of the second binding reagent may comprise a full-length protein, or a portion of a full-length protein sufficient to bind to the analyte.

The reporter domain of the second binding reagent comprises a detectable moiety that may be used to detect the presence of the second binding reagent. The detectable moiety may be any polypeptide or protein that is capable of detection, either directly or indirectly. Many such moieties are known. In a preferred embodiment, the detectable moiety is a detectable enzyme, such as luciferase, horseradish peroxidase, alkaline phosphatase, etc. Renilla luciferase (abbreviated herein as “Ruc”) is particularly preferred. In another embodiment, the detectable moiety comprises multiple copies of a detectable enzyme. Such may be accomplished by, for example, the use of a cloning vector coding for multiple copies of the enzyme, which may be linked in tandem, or located on either side of the binding domain. Other detectable moieties include, for example, fluorescent proteins such as green fluorescent protein, and various other colored proteins sold by, e.g., Clontech in their Living Colors™ product line.

The methods of the present invention may be used to detect a wide range of analytes, including, but not limited to, proteins, antibodies, carbohydrates, lipids, etc. It will be apparent that the analyte to be detected should be capable of binding simultaneously to the first and second binding reagents. In a preferred embodiment, the analyte is an antibody (e.g., an IgA, IgE, IgG, IgM, etc.), and the first binding reagent and the binding domain of the second binding reagent are both antigens. If the analyte is other than an antibody, then the first binding reagent and the binding domain of the second binding reagent may be antibodies that bind to the analyte.

The analyte to be detected may be indicative of the presence or progress of a disease state. For example, the present invention may be used detect the presence of antibodies generated in response to the presence of pathogens such as viruses, bacteria, fungi, parasites, etc. Any pathogen that generates a humoral response may be detected according to the present invention. A non-exhaustive list of pathogens is available on the American Biological Safety Association website (www.absa.org/resriskgroup.html). Particularly preferred viruses including their subtypes are HIV, CMV, Hepatitis B, Hepatitis C, West Nile virus, HPV, RSV, herpes, HTLV-1, SARS, etc. Particularly preferred bacteria include M. Tuberculosis, H. pylori, anthrax, F. tularensis, streptococcus, pneumococcus, E. coli, Clostridia, staphylococcus, meningococcus, the causative agents of various sexually-transmitted diseases such as syphilis and gonorrhea, the causative agents of Legionnaire's and Lyme disease, etc. Particularly preferred fungi/yeast include Pneumocystis, Candida, Saccharomyces, Histoplasma, Cryptococcus, and Aspergillis. Particularly preferred parasites/protozoa include Plasmodia, Schistosoma, Cryptosporidium, and Toxoplasma.

The present invention may also be used to detect antibodies generated in response to autoimmune diseases. Many such diseases are known, and include, for example, Alopecia greata, Antiphospholipid syndrome, Addison's disease, Arthritis, Ankylosing spondylitis, Dermatomyositis, Fibromyalgia-Fibromyositis, Juvenile arthritis, Polymyalgia Rheumatica, Polymyositis, Raynaud's phenomenon, Reiter's syndrome, Rheumatoid arthritis, Scleroderma, Sjogren's syndrome, Arteritis, Polyarteritis nodosa, Takayasu Arteritis, Temporal arteritis/Giant Cell arteritis, Autoimmune hemolytic anemia, Autoimmune hepatitis, Behcet's disease, Cardiomyopathy, Celiac Sprue, Celiac Sprue-dermatitis, Chronic Fatigue Immune Dysfunction Syndrome, Chronic Inflammatory Demyelinating Polyneuropathy, Churg-Strauss Syndrome, CREST syndrome, Cold Agglutinin Disease, Crohn's disease, Type 1 diabetes, Essential Mixed Cryoglobulinemia, Glomerulonephritis, Graves' disease, Guillain-Barre syndrome, Miller-Fisher syndrome, Idiopathic Pulmonary Fibrosis, Idiopathic Thrombocytopenia Purpura, IgA Nephropathy (Berger's disease), Inflammatory bowel disease, Lichen Planus, Lupus, Lupus nephritis, Systemic lupus erythematosis, Meniere's disease, Mixed connective tissue disease, Multiple sclerosis, Myasthenia gravis, Myocarditis, Pemphigus/pemphigoid, Bullous pemphigoid, Cicatricial pemphigoid, Pemphigus vulgaris, Pernicious anemia, Polychondritis, Polyglandular syndromes, Primary Agamma-globulinemia, Primary biliary cirrhosis, Psoriasis, Retinitis, Rheumatic fever, Sarcoidosis, Stiff-Man syndrome, Thyroiditis, Ulcerative colitis, Uveitis, Vasculitis, Vitiligo, and Wegener's granulomatosis.

The present invention may also be used to detect antibodies generated in response to chronic inflammatory diseases. Many such diseases are known, and include, for example, rheumatoid arthritis, osteoarthritis, chronic obstructive pulmonary disease, etc. The present invention may also be used to detect allergic reactions.

The present invention may also be used to detect antibodies generated in response to the presence of known tumor-associated proteins, such as p53, K-Ras, c-Myc, β-catenin, Smad4, PSA, etc., and hence may be used to detect or follow the course of various cancers, such as colon, breast, prostate, head and neck, etc. In that embodiment, the binding domain of the second binding reagent will comprise the tumor-associated protein, or at least a portion thereof sufficient to bind to the antibodies to be detected, e.g., the antigenic portion of the protein. Any cancer is contemplated to be within the scope of the present invention; a non-exhaustive list may be found at http://www.cancer.gov/cancertopics/alphalist, incorporated herein by reference. The present invention may also be used to detect or quantify the presence of proteins associated with a particular disease state, such as the tumor-associated proteins mentioned above.

Because the present invention is capable of quantifying an analyte, it will be readily apparent that the present invention may be used not only to detect the presence of a disease state, but also to monitor the progress of a disease or condition, and to monitor the progress of treatment of a disease or condition. In that instance, the present assay will be repeated over time to determine the change, if any, of the concentration of the particular analyte over time. Thus, the effectiveness of, e.g., a course of cancer therapy (chemotherapy, radiation, etc.) or infectious disease drug therapy may be determined readily.

The assay of the present invention may be performed by contacting the first binding reagent, the sample and the second binding reagent. If the analyte is present, a complex is formed among the two binding reagents and the analyte. Because the first binding reagent is immobilized, the second binding reagent likewise becomes immobilized if the analyte is present. The immobilized complex is separated from the reaction mixture, for example by washing, and the presence of the second binding reagent in the complex is detected via the reporter domain. The presence (or concentration) of the second binding reagent in the immobilized complex is indicative of the presence (or concentration) of the analyte. The order of addition of the reagents and analyte is not critical. Thus, the analyte may be mixed with the first binding reagent, then the second binding reagent may be added to the mixture. Alternatively, the steps could be reversed, i.e., the analyte may be mixed with the second binding reagent, then the first binding reagent may be added to the mixture. Finally, the present invention contemplates that the first and second binding reagents and the analyte could be mixed together at the same time, or the first and second binding reagents are pre-mixed and the analyte added to the mixture.

It will be apparent that the assay of the present invention may be carried out so as to detect a single analyte, e.g., by testing a sample with a single pair of binding reagents designed to detect a single analyte of interest. It is also contemplated that the present invention may be used to detect multiple analytes in a single sample. That may be done by utilizing multiple pairs of binding reagents designed to detect multiple analytes of interest. Such may be easily achieved by the use of, for example, multiple well plates, wherein the multiple first binding reagents are immobilized in discrete wells in the plate. Alternatively, multiple binding domains, each of which binds the different analytes, may be incorporated with the record binding reagent, for example in tandem.

The present invention also contemplates that the reagents for use in the assays described herein be contained in the form of a kit. Such kits would include, contained within suitable packaging material, an immobilized first binding reagent and a second binding reagent, both as described above. The kits may optionally further contain other components, such as instructional material for use of the kit, reagents for detecting the reporter domain of the second binding reagent (e.g., buffers, compounds that produce light when contacted with the detectable enzyme, etc.), a positive control for comparative or calibration purposes, etc.

The present invention will now be illustrated with reference to the non-limiting examples described below.

### EXAMPLE 1

We used Ruc-tagged antigen-fusion proteins to develop an immunoprecipitation assay that can quantitatively measure serum antibody reactivity with protein antigens. In brief, crude extract containing the Ruc-antigen fusions, sera and protein A/G beads are mixed together and incubated, during which the antigen fusions become immobilized; antigen-specific antibody is then quantitated by washing the beads and adding the colenterazine substrate. In these assays the amount of light produced is proportional to the amount of soluble fusion protein captured by the antibody-bound beads. It should be noted that the binding capacity of the protein A/G beads (Pierce Biochemical) used to capture either purified monoclonal antibodies or immunoglobulins from crude human or animal antisera is high (24 μg of immunoglobulins/μl of packed beads).

The Immunoprecipitation Assay Shows a Linear Range of Detection with Commercial Antibodies

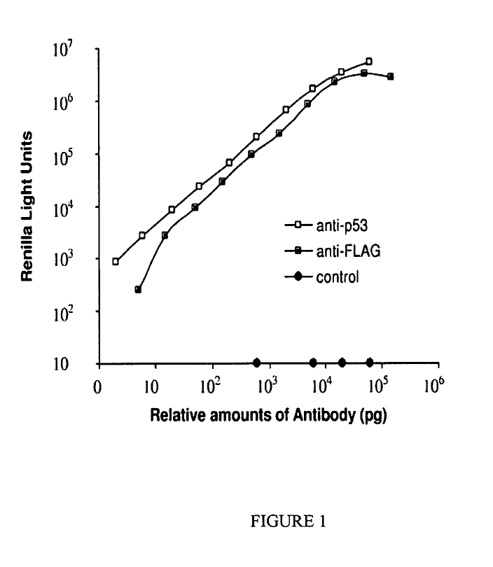

To illustrate this technology we generated Ruc fusion protein constructs for p53, K-Ras, c-Myc, β-catenin and Smad4 by fusing cDNAs encoding these proteins (in frame) to DNA encoding the C-terminus of Ruc in a mammalian expression vector, pREN2, which also encodes a FLAG epitope tag at the N-terminus of Ruc. Transfections into Cos1 cells of these different constructs yielded crude extracts with 3-10×10⁸ Ruc light units per 100 mm² plate. We developed a standard assay format by using a commercial anti-FLAG monoclonal antibody and Cos1 cell extracts containing Ruc-p53. When crude extract, antisera and protein A/G beads were incubated together in a single tube, the amount of immunoprecipitated Ruc-p53 was directly proportional to the amount of anti-FLAG antibody over a 1000-fold range of concentrations, with a lower limit of detection of less than 5 picrograms (FIG. 1). A commercial anti-p53 polyclonal antibody had a similar capture capacity as reflected by a similar dose-response curve, whereas a commercial polyclonal antibody against an unrelated antigen was unable to immunoprecipitate Ruc-p53 (FIG. 1). Experiments using commercial polyclonal antibodies for serine-15 phosphorylated p53 and acetylated p53 (lysine-373 and lysine-382) also immunoprecipitated significant amounts of Ruc-p53 (data not shown). Since the ability of these modification-specific antibodies to immunoprecipitated Ruc-p53 was not competed by bacterially-expressed recombinant p53 protein (data not shown), this fusion protein appears to contain at least two types of post-translational modifications.

Human Cancer Patient Sera Contain Antigen-Specific Antibodies

Since commercial antibodies can immunoprecipitate Ruc-antigen fusions from crude Cos1 extracts, we tested whether our simple assay format could also detect antigen-specific antibodies in clinical sera samples. Our motive for developing this technique was to have an improved method for detecting cancer patient antibody responses to tumor-associated proteins. Thus, we initially tested the assay with a small number of clinical sera samples taken from patients having three types of cancers: breast, colon and head and neck. In order to maximize our chances of detecting positive responses with these clinical sera samples we chose to use p53 and four other tumor-associated proteins (K-Ras, c-Myc, β-catenin and Smad4) that are either frequently mutated and/or overexpressed in various tumors. Wild-type proteins were used as antigens because several studies show that cancer patient sera humoral immune responses are not restricted to or even preferential for the epitopes that usually contain the altered amino acids [16, 18-21]. Cos1 extracts containing Ruc-antigen fusions were used to test a total of 36 sera, comprised of 10 controls and 26 cancer patients (Table 1). Negative and positive controls consisting of protein A/G beads alone and 0.1 μg of anti-FLAG monoclonal antibody with protein A/G beads, respectively, were used for each experiment. As expected, all sera had low reactivity with the non-specific binding control protein, Ruc-alone (Table 1). The positive control, anti-FLAG antibody, immunoprecipitated significant amounts of each of the Ruc-antigen fusions. However, the fraction of the total Ruc activity that could be captured varied amongst the different Ruc-antigen fusions, possibly reflecting reduced accessibility to the N-terminal FLAG epitope in some constructs (data not shown). At least one cancer patient sera had statistically significant antibody responses to each of the five Ruc fusions, where significance is defined as a response greater than the average plus three standard deviations of the 10 control sera (Table 1). Two of 10 head and neck, five of 10 breast, five of six colon cancer sera, but none of 10 healthy control sera gave positive responses. Six of the 12 positive tests were clustered in the six colon cancer patient sera and two antigens, p53 and K-Ras (Table 1). The significance of the relative response rates between different cancer-type sera cannot be calculated because the sample sizes are small and because no effort was made to match the control and patient sera by any criteria. Similarly, we cannot conclude that either K-Ras and/or p53 may be more antigenic in colon cancers than either β-catenin or c-Myc. Interestingly, the only multiple sample from any of the patients, head and neck samples 11 and 12, are sequential samples of which only the more recent sample showed significant levels of anti-p53 antibodies. Since the proteins used to test for antibodies in these 26 cancer patient sera are often mutated and/or overexpressed in the three types of cancer, our results are consistent with studies indicating that these categories of proteins are often antigenic in cancer patients [2]. Our results with colon cancer patient sera also indicate that humoral immune responses to panels of tumor-associated antigens may be clinically useful when single antibody responses are not informative [22, 23].

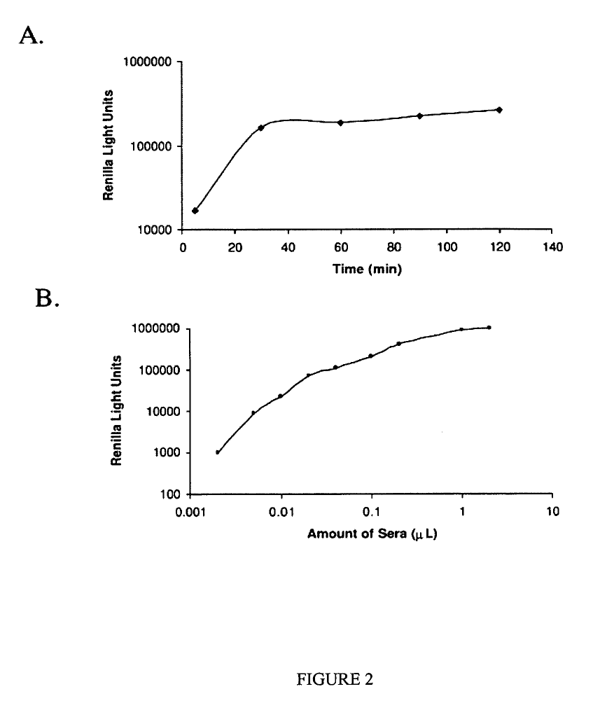

To determine whether patient antibody responses behave in the same linear manner as the commercial antibodies, we used the most reactive combination of patient sera and fusion antigen in our sample set, colon cancer sera 34 and the Ruc-p53 fusion. Although the amount of Ruc-p53 captured by this serum is roughly linear with incubation time in the presence of protein A/G beads, reaching a plateau by 30-60 minutes (FIG. 2A), the relative amount of immunoprecipitated Ruc-p53 was not completely linear with increasing amounts of sera (FIG. 2B). Since the two commercial antibodies used in FIG. 1 are highly purified, the non-linear dose-response curve of the clinical sera sample could be due to interfering agents such as anti-p53-specific IgA and IgM antibodies that recognize epitopes also recognized by IgG's but which bind poorly to protein A/G beads [24].

Competition Experiments with Unmodified Proteins

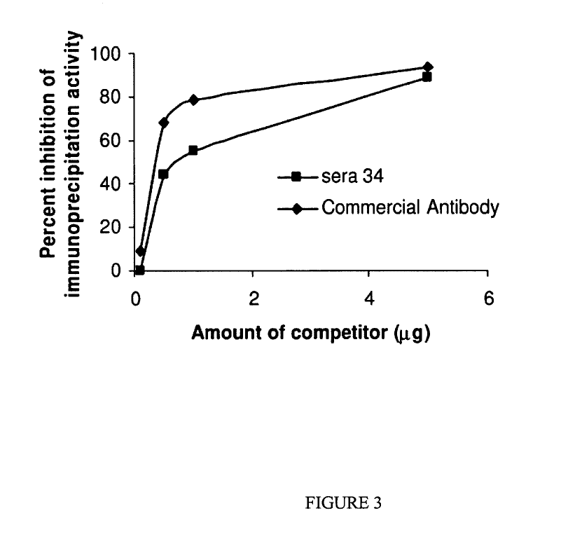

While human humoral immune responses to post-translational modifications are often ignored and/or undetectable with existing technologies, recent studies demonstrate that disease-related antibody responses can occur to post-translational protein modifications [25]. In at least one case, rheumatoid arthritis, antibody responses to a post-translational modification, citrullination, is now being intensely investigated as a potentially reliable disease indicator [26,27]. In light of these observations, we asked whether each positive sera response seen in Table 1 could include antibodies that were directed toward post-translational modifications by doing competition experiments with unmodified E. coli-produced antigens. These competition experiments (Table 2) show that 0-100% of the immunoprecipitated Ruc-antigen fusions were blocked by preincubating sera with 5 μg of the corresponding E. coli-produced antigens fused to maltose binding protein (MBP). These differences occur even between sera containing antibodies that recognize the same antigen (e.g. p53 or K-Ras), proteins known to contain post-translational modifications. These differences could mean that some tumors tend to produce proteins having more post-translational modifications or that some cancer patients' immune systems tend to produce significantly more antibodies that recognize post-translational modifications. However, this data does not exclude the possibility that some or all of each positive antibody response detected is not even specific for the antigen listed, since the apparent anti-p53 or anti-K-Ras antibodies could be directed toward proteins that are in complexes with these tumor antigens. If the tumor antigens in these complexes were easily replaced by the MBP fusions, one would see higher competition values than if they were inefficiently replaced. Quantitative evaluation of different competition results requires, at a minimum, equal amount of reactive antibodies in each sera, a condition unlikely to be satisfied here, especially for the p53-reactive sera. In addition, when we compared the dose-response competition curves of sera 34 and the commercial polyclonal anti-p53, adjusted to similar capacities for immunoprecipitating Ruc-p53, we found a greater difference than indicated by the end-point values alone (FIG. 3). Nevertheless, it is clear that the present assay identifies patient sera having qualitatively different humoral immune responses to the same antigen.

These data show that our approach of making antigen-enzyme fusions and producing these fusions in mammalian cells is superior to conventional ELISA assays for detecting antigen-specific antibody responses in human sera. Specifically, we have tested the six colon cancer patient sera used here in a standard sandwich type ELISA where the antigen were fused to E. Coli MBP and immobilized on ELISA plates with a monoclonal anti-MBP antibody. In these ELISA tests only two of the six colon cancer sera gave positive responses with any of the five tumor-associated proteins listed in Table 1 (data not shown). In any case, the immunoprecipitation assay described here offers a practical approach for identifying post-translational modification-specific antibody responses and studying their medical relevance.

Quantitative results were obtained by using easily prepared crude cell extracts containing post-translationally modified antigens fused to a light-producing enzyme reporter. While the immunodetection of antigen-enzymes is not new [28, 29], by combining a robust reporter, such as Ruc with the production of recombinant enzyme-antigen fusions in mammalian cells, we have created a highly sensitive, user friendly assay. This assay requires fewer manipulations for reagent preparation and less time than other immunoprecipitation methods including avoiding having to purify and then radiolabel the purified proteins or having to perform additional analysis such as Western blotting after the immunoprecipitations [30]. Thus, it is within the scope of the present invention to utilize the second binding reagent in less than completely pure form, i.e., as a component of a crude extract. Producing the target antigens in mammalian cells offers several potential advantages, including having mammalian-specific and/or disease-specific post-translational modifications added to these antigens. Thus, this immunoprecipitation assay provides a simple, accessible, reliable and reproducible tool for investigations aimed at documenting the role of post-translational modification in disease. Although altered post-translationally modified proteins occur in cancer [31, 32], future studies are needed to explore whether there are detectable cancer patient-specific antibodies to post-translationally-modified tumor proteins. The levels and kinds of post-translational modifications on the Ruc-antigen fusions can be manipulated by exploiting mutant proteins, unique human cell lines (e.g. cell lines overexpressing tyrosine kinases) and various culture conditions. Mammalian-produced antigens have additional advantages over bacterial-expressed antigens including facilitating the study of antibody responses to very large proteins (>100 kDa) that are difficult or impossible to produce as intact proteins in E. coli. Our assay also avoids false positives caused by variable amounts of anti-E. coli antibodies present in patient sera that react with the minor amounts of E. coli proteins that co-purify with bacterial recombinant proteins; such contaminants are even present in some pharmaceutical-grade recombinant protein preparations [3]. These advantages, along with the possibility of improving the assay format, make this assay worthwhile to reevaluate the frequency with which known tumor-associated proteins are detectably antigenic in cancer patients. It is encouraging, although of limited significance, that the frequencies of significant antibody responses for two of the cancers are roughly comparable to reports in the literature. Thus, in colon cancer patients we detected statistically significant antibody responses to Ras and p53 in 50% and 33% of the sera, respectively, compared to published reports of 33% for Ras [1,6] and 26% for p53 [33]. In contrast, we did not find any statistically significant antibody responses to p53 in breast cancer sera, which have been reported to occur with 9% of patient sera [34].

This assay format and high-throughput modifications (e.g. magnetic A/G beads in a microtiter plate format) are obviously directly applicable to detecting human sera antibodies specific for any protein antigen of interest and is also useful for non-human sera, such as sera obtained from animal models of disease, as well as for antibodies in other bodily fluids including from urine, cerebrospinal fluid, amniotic fluid, gastic fluid, ascites and saliva. Variations of this immunoprecipitation assay format might also be useful for studying other types of protein-protein interactions.

Methods

Biochemical Reagents and Antibodies

Ultralink™ immobilized protein A/G beads were obtained from Pierce Biotechnology Inc. Commercially available antibodies were: mouse monoclonal anti-FLAGT™ M2 from Sigma; rabbit anti-acetylated p53 from Upstate Biochemicals and polyclonal rabbit anti-p53, polyclonal rabbit phosphoserine p53 and polyclonal anti-WASP from Santa Cruz Biotechnology.

Patient Sera

The breast and colon cancer patient sera were obtained from the University of Wisconsin collection, now kept at Georgetown University Medical Center. Sera samples from head and neck cancer patients and control sera were collected by Dr. Radoslav Goldman at Georgetown University Medical Center (Washington, D.C.). The sex, age and disease stages of these samples were not examined until after the reactivities for all antigens were measured.

Generation of Constructs Encoding Ruc Fused to Tumor-Associated Antigens

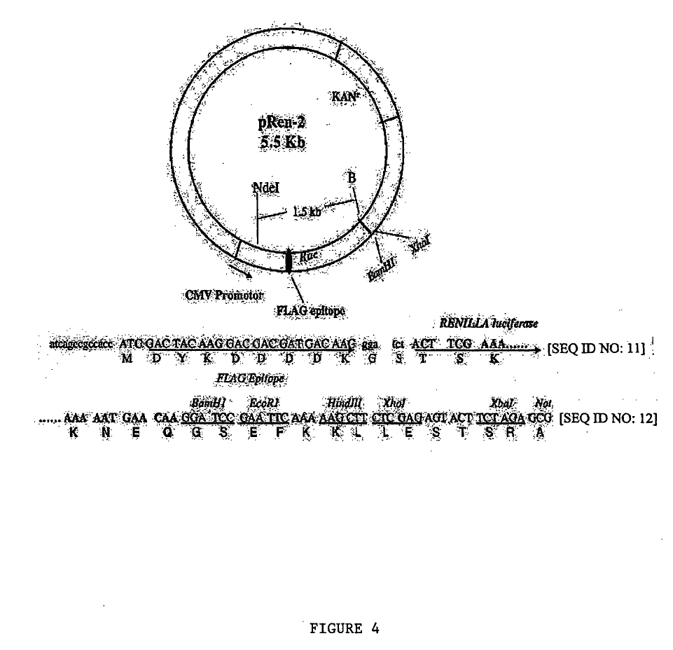

pREN2, a FLAG-epitope-tagged mammalian expression vector, similar to the previously described pREN1 [4], was used to generate all plasmids encoding Ruc fusions. The tumor antigens are at the C-terminus and a single FLAG tag is at the N-terminus of Ruc. A map of pREN2 is shown in FIG. 4. The cloned human cDNA fragments, amplified by PCR specific linker-primer adapters, were obtained from Dr. E. Chang (p53), Dr. R. Lechleider (Smad4), Dr. S. Byers (β-catenin), Dr. R. Dickson (c-Myc) and a publicly available cDNA clone (IMAGE ID 6714574) for K-Ras. Full-length coding sequences (excluding the initial methionine) were used for the tumor antigens, with the exception of the β-catenin, which encodes amino acids 2-277. In every case a stop codon was included after the C-terminal coding sequences of the tumor antigens. The primer adapter sequences used for cloning each antigen are as follows:

p53:

[SEQ ID NO: 1]

5′-GAGGGATCCGAGGAGCCGCAGTCAGAT-3′
and

[SEQ ID NO: 2]

5′-GAGCTCGAGTCAGTCTGAGTCAGGCC-3′;
K-Ras:

[SEQ ID NO: 3]

5′-GAGGGATCCACTGAATATAAACTTG TG-3′
and

[SEQ ID NO: 4]

5′-GAGCTCGAGTTACATAATTACACACTT;
Smad4:

[SEQ ID NO: 5]

5′-GAGGGATCCGACAATATGTCTA TTACG-3′
and

[SEQ ID NO: 6]

5′-GAGCTCGAGTCAGTCTAAAGGTTGTGG-3′;
β-catenin-Δ:

[SEQ ID NO: 7]

5′-GAGGGATCCGCTACT CA AGCTGATTTG-3′
and

[SEQ ID NO: 8]

5′-GAGGTCGACTCAACCAGCTAAACGCACTGC-3′;
and
c-Myc:

[SEQ ID NO: 9]

5′-GAGG GATCCCTCAACGTTAGCTTCACC-3′
and

[SEQ ID NO: 10]

5′-GAGCTCGAGTTACGCACAAGAGTTCCG-3′.

For Ruc alone, a separate construct was prepared containing a stop codon at the end of the luciferase coding sequence in place of the polylinker present in pREN2.
Immunoprecipitation Assays with Ruc Fusion Proteins

Forty-eight hours after Fugene-6 transfection, Cos1 cells in 100 mm² plates were washed twice with PBS, scraped with 1.0 ml of Buffer A (20 mM Tris, pH 7.5, 150 mM NaCl, 5 mM MgCl₂, 1% Triton X-100) plus 50% glycerol and protease inhibitors (10 μg/mL each of leupeptin, aprotinin and pepstatin), sonicated, centrifuged at 13,000×g for 4 min, supernatants collected and used immediately or stored at −20° C. Total luciferase activity in 1 μl of each crude extract was measured by adding it to 100 μl of assay buffer and substrate mixture (Renilla Luciferase Reagent Kit, Promega) in a 12×75-mm glass tube, vortexing and immediately measuring light-forming units with a luminometer (GeneProbe) for 10 sec. Lysate prepared from each 100 mm² plate of transfected Cos1 cells typically provides enough extract for 60-200 assays. These crude Cos1 extracts containing these Ruc fusions were stable for at least a few weeks when stored in 50% glycerol at −20° C.

Immunoprecipitation assays were performed in 100 μl volumes containing 6 μl of a 30% suspension of protein A/G beads (in PBS), 1-10 μl sera (undiluted or diluted in Buffer A plus 100 μg/ml BSA), sufficient Cos1 cell extract to generate 1-5 million light units (usually 5 μl to 10 μl) and Buffer A and incubated at 4° C. with tumbling for 5-120 minutes, washed 4-5 times with 1.2 ml of cold Buffer A and once with 1.0 ml of PBS. After the final wash, the beads, in a final volume of about 10 μl, were added to the Ruc substrate and light units measured as described above. Since the capacity of these protein A/G beads is 24-32 mg/ml of packed beads, 2 μl of packed beads should be sufficient to immobilize most or all of the IgG in 1 μl of undiluted sera (assumed to be 10 mg/ml IgG). The amount of IgG in 2 μl of each sera that actually bound to protein A/G beads was estimated by measuring the amount of bead-bound sera released by a low pH glycine elution buffer and measured using the BCA Protein Assay kit (Pierce Biotechnology Inc.). The protein values varied from 2.0 to 7.3 μg/μl of patient sera.

Competition experiments were performed using MBP-fusion proteins. Bacterial expression vectors were constructed by subcloning cDNA fragments into the pMAL-c2 vector (New England Biolabs). Recombinant MBP fusion proteins were produced in bacteria, purified by amylose-agarose affinity and eluted with maltose as described by the manufacturer and stored frozen or in 50% glycerol at −20° C. An MBP fusion containing the SPEC2 cDNA [35] was produced and used as a non-specific inhibitor. The integrity of the proteins was confirmed by SDS-PAGE electrophoresis and protein concentration determined. Diluted patient sera (10 μl used of sera diluted 1:10 in buffer A containing 100 μg/ml BSA) were used in the competition experiments described in Table 2, while only 5 μl of 1:10 diluted colon patient sera 34 was used in the experiments described in FIG. 3.

TABLE 1
Immunoprecipitation capacity of 1 μl of human
sera for Ruc-tumor antigen fusion proteins^{a}

| | | Ruc | p53 | K-Ras | Smad4 | β-CAT-Δ1 | c-Myc |
|---|---|---|---|---|---|---|---|
| Controls | 1 | 194 | 19,319 | 480 | 10,582 | 269 | 4,752 |
| | 2 | 9 | 9,830 | 1,064 | 3,575 | 835 | 2,913 |
| | 3 | 8 | 5,236 | 445 | 1,773 | 211 | 2,006 |
| | 4 | 38 | 3,187 | 477 | 1,919 | 530 | 1,831 |
| | 5 | 14 | 11,908 | 795 | 6,884 | 161 | 3,346 |
| | 6 | 31 | 5,390 | 823 | 1,724 | 235 | 2,050 |
| | 7 | 76 | 22,526 | 1,909 | 6,996 | 259 | 11,816 |
| | 8 | 29 | 15,338 | 943 | 8,043 | 445 | 3,475 |
| | 9 | 10 | 12,282 | 1,162 | 19,380 | 215 | 3,623 |
| | 10 | 9 | 11,130 | 1,109 | 4,429 | 501 | 5,060 |
| x̅ + 3 SD^{b} | | 214 | 30,234 | 2,237 | 22,788 | 997 | 12,874 |
| Head | 11 | 0 | 10,904 | 508 | 2,721 | 196 | 2,193 |
| and Neck | 12 | 0 | 31,593^{c} | 738 | 4,822 | 465 | 3,801 |
| | 13 | 0 | 12,367 | 840 | 1,868 | 673 | 4,407 |
| | 14 | 13 | 14,705 | 1,012 | 5,666 | 195 | 1,837 |
| | 15 | 33 | 31,733^{c} | 1,189 | 5,264 | 552 | 4,107 |
| | 16 | 121 | 4,828 | 621 | 980 | 279 | 1,974 |
| | 17 | 0 | 8,517 | 1,160 | 8,396 | 336 | 2,958 |
| | 18 | 0 | 19,240 | 1,283 | 9,485 | 327 | 1,814 |
| | 19 | 0 | 11,224 | 1,517 | 4,454 | 410 | 4,370 |
| | 20 | 28 | 7,322 | 554 | 2,261 | 723 | 2,343 |
| Breast | 21 | 44 | 13,211 | 960 | 10,219 | 308 | 5,988 |
| | 22 | 10 | 18,814 | 696 | 42,970^{c} | 302 | 5,450 |
| | 23 | 38 | 14,598 | 608 | 8,484 | 339 | 4,336 |
| | 24 | 77 | 11,587 | 1,655 | 17,297 | 2363^{c} | 3,431 |
| | 25 | 17 | 19,954 | 532 | 10,184 | 772 | |
| | | | | | | | 15,650^{c} |
| | 26 | 25 | 9,538 | 195 | 5,962 | 300 | 1,646 |
| | 27 | 10 | 7,815 | 2,561^{c} | 20,628 | 426 | 3,524 |
| | 28 | 21 | 15,607 | 308 | 7,380 | 284 | 1,579 |
| | 29 | 0 | 18,058 | 160 | 6,790 | 304 | 2,333 |
| | 30 | 245^{c} | 25,479 | 1,919 | 9,727 | 495 | 3,787 |
| Colon | 31 | 4 | 6,656 | 1,204 | 3,252 | 267 | 1,763 |
| | 32 | 40 | 20,928 | 4,293^{c} | 5,567 | 962 | 6,143 |
| | 33 | 42 | 34,703^{c} | 1,472 | 10,830 | 716 | 4,906 |
| | 34 | 51 | 300,943^{c} | 6,439^{c} | 2,610 | 992 | 3,789 |
| | 35 | 35 | 5,670 | 3,306^{c} | 3,860 | 477 | 1,772 |
| | 36 | 44 | 6,516 | 695 | 37,344^{c} | 371 | 2,395 |
| ^{a}Sera, FLAG-Ruc-fusion extracts, protein A/G beads and buffer were mixed together, incubated for 60 minutes and processed. The data, light units, is the average of two experiments and is corrected for background (beads plus extract, but no sera). The standard | | | | | | | |
| # deviation for each value is also available (see Additional file 1). | | | | | | | |

^{b}Values of the averages of the 10 control sera plus 3 standard deviations.
^{c}Numbers in bold are statistically significant: greater than the average plus 3 standard deviations of the 10 control sera.

TABLE 2
Competition of antibody responses by unmodified antigens^{a}

| Antigen/ | | | | | | |
|---|---|---|---|---|---|---|
| sera | Control | p53 | K-Ras | Smad4 | β-CAT-Δ1 | c-Myc |
| P53/12 | 21% | 32% | | | | |
| P53/15 | 20% | 60% | | | | |
| P53/33 | 7% | 88% | | | | |
| P53/34 | 11% | 72% | | | | |
| K-Ras/27 | 5% | | 91% | | | |
| K-Ras/32 | 25% | | 82% | | | |
| K-Ras/34 | 4% | | 0% | | | |
| K-Ras/35 | 16% | | 100% | | | |
| Smad4/22 | 4% | | | 92% | | |
| Smad4/36 | 0% | | | 93% | | |
| β-catenin- | 23% | | | | 96% | |
| Δ1/24 | | | | | | |
| c-Myc/25 | 0% | | | | | 22% |
| ^{a}Sera (1 μl), buffer and 5 μg competitor were incubated together for 60 min before adding the fusion | | | | | | |
| # extracts and protein A/G beads for an additional 60 minutes and processed. Background light units (beads plus extract but no sera) were | | | | | | |
| # subtracted before calculating percent competition. The first column identifies | | | | | | |
| # the antigen-sera combination tested. The other columns give the amount of competition obtained for each competitor | | | | | | |
| # antigen. All competitors, including the control (SPEC2), are MBP fusion proteins. Values are the averages plus from two independent | | | | | | |
| # experiments. The standard deviation for each value is also available (see Additional file 2). | | | | | | |

### EXAMPLE 2

Results

Rapid and Accurate Identification of Human Sera Containing Anti-HIV Antibodies Using an Antigen-Reporter Fusion Protein as the Antigen

We reported the successful use of antigen-Renilla luciferase (Ruc) fusions, produced in Cos1 monkey cells, in a simple immunoprecipitation assay, to quantitatively measure human serum antibody responses to tumor-associated proteins (38). Here, we tested whether a minor modification of this technology could be used to successfully predict the infection status of blinded serum samples, some of which were from patients exposed to infectious agents.

Serum antibody responses to HIV were measured by using a single protein antigen, a fusion between a major HIV core protein, p24, and Renilla luciferase. Transient transfections of Cos1 cells with an expression vector for this fusion yielded crude extracts able to generate 2-10×10⁸ Ruc light units per 100 mm² plate, similar to the amounts of Ruc light units obtained with other human Ruc-antigen fusions produced under similar conditions (38). Using one such crude extract as the antigen, we profiled 28 sera, consisting of equal numbers of previously characterized HIV positive and negative sera, although we did not know at the time of testing that this set contained equal numbers of positive and negative sera. The methods used to classify these sera as positive or negative (Western Blotting and/or PCR) were also unknown to us (at Georgetown University) at the time of our tests. We tested these sera at two different times, about 6 days apart, using two different crude extracts, both of which were stored at −20° C. before use. For this test we slightly modified our previously described assay format (38). Instead of combining sera, antigen and protein A/G beads all together and then incubating, we first combined and incubated only the sera and antigen and then subsequently added protein A/G beads, under the theory that antigen-antibody complex formation is more likely to be limiting than antibody immobilization onto the beads. That is, antigen-antibody complexes will form more rapidly in solution than after antibody immobilization onto the beads and that quantitative immobilization of antibodies onto the beads is likely to be independent of whether or not they have already formed antibody-antigen complexes. In any case, this modified assay format yielded quantitative values, whose averages span almost four-logs and which probably reflect relative antibody titers (Table 3). When tabulated from lowest to highest, it was believed that that this blinded serum set contained 14 HIV positive and 14 HIV negative sera, which turned out to be correct. The 14 lowest values were less than 1000, with an average of 309 and a relatively small standard deviation of 126. The average of the highest 14 values is 595,300 with a standard deviation of 1,091,000. Clearly, the highest 14 values vary considerably more than the lowest 14, a result expected if our classification was correct and if the 14 highest values are indeed positive sera and are from individuals in different states of disease progression (a speculation also confirmed after the blinded code was broken, see below). Breaking the blinded code confirmed both of our predictions. Thus, this initial blinded assay for HIV yielded 100% sensitivity and 100% specificity.

Additional clinical information about these sera revealed that four of the weakest, but still positive responders, with average titers of 2,716, 4,785, 8,507 and 9,317 light units, were obtained from chronically HIV-infected individuals. These four low anti-p24 antibody titers, in sera from chronically infected individuals, are consistent with several studies showing that anti-p24 HIV antibody responses are lost during disease progression (36; 47) or following successfully antiviral therapy (42). It should be noted that this successful classification was accomplished with crude extracts, was done without optimizing assay conditions and did not involve the use of a training sera set to determine cut-off values. In addition, each set of 28 assays required relatively short hands-on time and less than 15 hours from mixing sera with antigen to data collection.

Comparisons Between the IP and ELISA Assays for Detecting Antibodies Against Pneumocystis jiroveci in Sera from Non-Infected and HIV-Infected Individuals

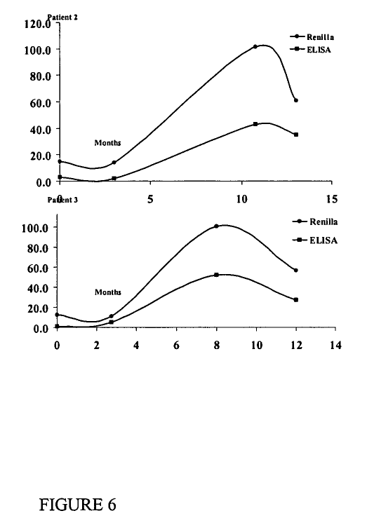

We next determined whether our immunoprecipitation assay could accurately reproduce the relative antibody titers to Pneumocystisjiroveci, a pathogen often associated with Pneumocystis pneumonia (PCP) in immunocompromised individuals. All of the current ELISA assays used to detect antibodies to Pneumocystisjiroveci employ only fragments of MSG-14, a large major surface glycoprotein of Pneumocystisj. (37; 39) and these fragments are produced in bacteria. Using a crude Cos-1 Ruc-MSG-14Ca fusion extract as the antigen in our immunoprecipitation assay format, we blindly profiled the set of 28 sera already analyzed for anti-p24 HIV antibodies, described above. As shown in Table 3, the antibody responses to this antigen, measured in our immunoprecipitation assay, ranged from 4,318 to 1,260,205 light units. Following unmasking of the blinded samples, we compared the immunoprecipitation and ELISA assay values of the anti-MSG-14 antibody titers and found a high level of correlation (R=0.86), but only if sample #20 was omitted from the analysis (FIG. 6). The cause for this large discrepancy between the two assays methods with sample #20 is not known. One possibility is that the ELISA titer of serum #20 is artificially low; this serum had a larger ELISA signal with the negative control protein than any other sera in this set. Thus, repeating these ELISA assays with a different negative control protein may reveal a larger MSG-14-specific antibody titer. Another possibility is that sera #20 contains a high titer of antibodies against epitopes absent from the E. coli produced protein fragment, such as mammalian-specific posttranslational modifications or conformation-specific epitopes. This discrepancy is unlikely to be due to an artificially high titer in the immunoprecipitation assay caused by cross-reactivity of this serum with the Ruc portion of the fusion protein antigen. That is, this serum has a low non-specific background value in immunoprecipitation assays with Ruc-p24 HIV (Table 3) and with the Ruc-alone antigen control (data not shown). In addition, the range of positive values obtained in the two assays for anti-MSG-14 antibodies also differ, with the IP assay, even in its unoptimized form, having a larger dynamic range. These comparisons suggest that the immunoprecipitation assay may be more sensitive than commonly used ELISAs for a wide spectrum of antigen-specific antibodies. These immunoprecipitation assay results are encouraging and suggest that this simple assay can be rapidly used to profile patient humoral responses to this fungal pathogen.

Compare Methods for Monitoring P. jiroveci-Specific Antibodies in Serial Serum Samples

Antibody titer profiles generated from serial samples often reflect the clinical course of infection. To test whether the present assay is as good as an ELISA for detecting patient antibody responses to P. jiroveci in longitudinal sera samples, we analyzed, in a blinded fashion, a set of 24 samples collected from 6 patients at four time points over about a year spanning their P. jiroveci infections. A comparison of the antibody titer values obtained using the immunoprecipitation assay and a standard ELISA is shown in FIG. 6 and Table 4. The immunoprecipitation assay results track the ELISA assay results for each serum. At least four different profiles shapes were found with each assay method; always high, always low, higher early than late and higher late than early. These results, combined with the single time point data, suggest that our simple, quick, dirty (using crude extracts) and not-yet optimized immunoprecipitation assay is as accurate as an optimized ELISA for detecting and monitoring human humoral responses to P. jiroveci exposure.

TABLE 3

| | Ruc-p24 | Predicted | Known Clinical | Ruc-MSG14Ca |
|---|---|---|---|---|
| Sample | light units | HIV status | HIV Status | Light units |
| 2 | 161 | − | − | 116,714 |
| 3 | 18,045 | + | + | 14,048 |
| 4 | 3,810 | + | + | 63,318 |
| 6 | 379 | − | − | 38,128 |
| 7 | 3,231,998 | + | + | 188,912 |
| 9 | 703,287 | + | + | 6,778 |
| 12 | 274 | − | − | 254,202 |
| 15 | 357 | − | − | 65,530 |
| 17 | 335 | − | − | 322,645 |
| 18 | 110 | − | − | 6,619 |
| 19 | 17,185 | + | + | 31,730 |
| 20 | 2,716 | + | + | 1,260,205 |
| 23 | 8,507 | + | + | 42,239 |
| 24 | 109,053 | + | + | 18,035 |
| 28 | 9,317 | + | + | 12,005 |
| 29 | 598,893 | + | + | 199,241 |
| 30 | 267 | − | − | 156,811 |
| 31 | 2,957,536 | + | + | 332.265 |
| 35 | 4,785 | + | + | 15,782 |
| 36 | 312 | − | − | 22,620 |
| 40 | 372 | − | − | 24,101 |
| 43 | 451 | − | − | 771,501 |
| 45 | 53,789 | + | + | 175,218 |
| 48 | 614,856 | + | + | 134,150 |
| 50 | 276 | − | − | 82,293 |
| 51 | 601 | − | − | 27,151 |
| 54 | 170 | − | − | 4,318 |
| 56 | 255 | − | − | 18,816 |

TABLE 4

| | | Ruc-MSG-14Ca | |
|---|---|---|---|
| | ˜Month | light units (10³) | ELISA |
| Patient 1 | 0 | 12.3 | 2 |
| | 3 | 16.5 | 2 |
| | 6 | 12.4 | 4 |
| | 12 | 18.1 | 2 |
| Patient 2 | 0 | 12.7 | 1 |
| | 2.75 | 11.1 | 5 |
| | 8 | 100.4 | 52 |
| | 12 | 56.6 | 27 |
| Patient 3 | 0 | 14.8 | 3 |
| | 3 | 13.9 | 2 |
| | 10.75 | 101.4 | 43 |
| | 13 | 60.9 | 35 |
| Patient 4 | 1.75 | 453.7 | 299 |
| | 4.5 | 306.3 | 214 |
| | 7.25 | 28.4 | 8 |
| | 13.5 | 16.1 | 6 |
| Patient 5 | 0 | 16.8 | 4 |
| | 2.75 | 54.3 | 35 |
| | 7 | 100.0 | 196 |
| | 12.25 | 89.4 | 99 |
| Patient 6 | 0 | 119.8 | 43 |
| | 3 | 169.0 | 46 |
| | 6 | 179.2 | 39 |
| | 11 | 105.1 | 31 |

### EXAMPLE 3

The Immunoprecipitation Assays can Detect Antibodies to Multiple Components of Hepatitis C Virus (HCV) in Individual Human Sera

We investigated whether the present immunoprecipitation assay could detect antibody responses to multiple different proteins of HCV in single serum samples. A new, blinded set of 33 clinical sera samples, comprising unknown numbers of non-infected individuals and individuals infected with one or combinations of HBV, HCV and HIV were evaluated for antibody titers against Ruc-core, Ruc-NS3 fragment (C33) and Ruc-NS5A of HCV. The results of these HCV tests are given in Table 5. Based on the relatively sera titers and range of titers for each of the antigens, we predicted that there were 13 positives and 20 negatives for the HCV core, 10 positives and 23 negatives for NS5A and 10 positive and 20 negative for the NS3 fragment (C33). By combining the results for the three independent HCV assays we stipulate that a positive in any of the three HCV tests signals a positive sera test. If so, there are 13 positives and 20 negatives. Our classifications of the HCV status of these 33 samples completely agreed with their clinical status (Table 5). Thus, this initial blinded assay for HCV yielded 100% sensitivity and 100% specificity. Further comparisons show that the HCV core test correctly scored 13/13 positives (100%), the C33 test detected 12/13 positives (92.5%) and the NS5A test detected 10/13 positives (77%). An appealing aspect of this immunoprecipitation test is the large gap between the highest negative test value and the lowest positive test value for many of the antigens tested. Based on these promising results, it is apparent that determining the breadth, strength and kinetics of IgG antibody responses against the entire and/or partial proteome of HCV and other infectious agents is highly feasible and may have significant diagnostic and prognostic value.

Generation of Ruc-Antigen Fusion Constructs

pREN², a FLAG-epitope-tagged mammalian expression vector was used to generate all plasmids. DNA templates for various pathogens were obtained from Dr. J. Casey for HBV, Dr. C. Rhodes for HIV-1, and Dr. Pad Padmanabhan for the NS5A protein of HCV. The MSG-14 can clone of Pneumocystis j. was previously described (ref). Full-length coding sequences (excluding the initial methionine) were used for the tumor antigens, with the exception of the MSG-14 which encoded amino acids 2-277. In every case a stop codon was included after the C-terminal coding sequences of the tumor antigens. PCR specific linker-primer adapters were used for amplification. The primer adapter sequences used for cloning each antigen are as follows:

p24 HIV:

[SEQ ID NO: 13]

5′-GAGGGATCCCCTATAGTGCAGAACATC-3′
and

[SEQ ID NO: 14]

5′-GAGCTCGAGTCACAAAACTCTTGCCTTATG-3′;
MSG-14:

[SEQ ID NO: 15]

5′-GAGGGATCCACTGAATATAAACTTG TG-3′
and

[SEQ ID NO: 16]

5′-GAGCTCGAGTTACATAATTACACACTT-3′;
MSG-14:

[SEQ ID NO: 17]

5′-GAGGGATCCGATTTCGATCCAAC-3′
and

[SEQ ID NO: 18]

5′-GAGCTCGAGCTAAATCATGAACGAAATAAC-3′;
MSG14CA:

[SEQ ID NO: 19]

5′-GAGGGATCCTGTAATAAATGGTCTAGAAG-3′
and

[SEQ ID NO: 20]

5′-GAGCTCGAGCTACCCGCTCACCCTCAAG-3′;
HBV core:

[SEQ ID NO: 21]

5′-GAGGGATCCAGACATTGACCCTTATAAAG-3′
and

[SEQ ID NO: 22]

5′-GAGCTCGAGCTAACATTGAGATTCCG-3′;
HCV NS5A:

[SEQ ID NO: 23]

5′-GAGAGATCTTCCGGTTCCTGGCTAAGG
and

[SEQ ID NO: 24]

5′-GAGCTCGAGTCAGCAGCACACGACATCTTC-3′;
HCV NS3 (C33):

[SEQ ID NO: 25]

5′-GAGGGATCCGCGGTGGACTTTATCCCT-3′
and

[SEQ ID NO: 26]

5′-GAGTCTAGATCAACAGCTGTTGCAGTCTATC-3′.

Following construction, the different mammalian cell pREN2 expression vectors for the different antigens were purified using Qiagen Midi preparation kits.

TABLE 5
Detecting Patient Antibody Titers to
HCV Proteins and HCV Infection Status

| Sample | HCV | HCV | HCV | Predicted | Known HCV |
|---|---|---|---|---|---|
| Status | NS5A | Core | NS3 (C33) | HCV Status | Clinical |
| H1 | 860 | 1,016 | 1,576 | NEG | NEG |
| H2 | 49 | 226 | 1,878 | NEG | NEG |
| H3 | 2,602,015 | 521,175 | 3,346,890 | POS | POS |
| H4 | 562 | 600,560 | 1,722,455 | POS | POS |
| H5 | 909,591 | 196,452 | 3,254,280 | POS | POS |
| H6 | 748 | 855 | 1,963 | NEG | NEG |
| H7 | 1,188,895 | 1,557,117 | 787,366 | POS | POS |
| H8 | 0 | 351 | 1,688 | NEG | NEG |
| H9 | 0 | 1,158 | 1,667 | NEG | NEG |
| H10 | 0 | 0 | 1,948 | NEG | NEG |
| H11 | 0 | 0 | 1,198 | NEG | NEG |
| H12 | 2,540,682 | 481,090 | 5,808,141 | POS | POS |
| H13 | 0 | 1,058,123 | 8,106,051 | POS | POS |
| H14 | 173,925 | 606,811 | 537,546 | POS | POS |
| H15 | 0 | 2,707 | 2,383 | NEG | NEG |
| H16 | 0 | 2,537 | 2,043 | NEG | NEG |
| H17 | 14,687 | 8,525 | 1,830 | NEG | NEG |
| H18 | 266,415 | 554,962 | 1,575,607 | POS | POS |
| H19 | 594 | 469,759 | 3,248 | POS | POS |
| H20 | 0 | 5,053 | 1,379 | NEG | NEG |
| H21 | 0 | 7,316 | 1,268 | NEG | NEG |
| H22 | 0 | 4,893 | 2,647 | NEG | NEG |
| H23 | 0 | 5,350 | 2,870 | NEG | NEG |
| H24 | 775,821 | 751,113 | 5,654,370 | POS | POS |
| H25 | 812,030 | 1,009,521 | 5,052,565 | POS | POS |
| H26 | 0 | 1,973 | 1,577 | NEG | NEG |
| H27 | 38 | 7,757 | 1,824 | NEG | NEG |
| H28 | 646,586 | 109,9054 | 4,616,469 | POS | POS |
| H29 | 0 | 891 | 4,523 | NEG | NEG |
| H30 | 0 | 4,509 | 6,460 | NEG | NEG |
| H31 | 0 | 6,366 | 2,406 | NEG | NEG |
| H32 | 0 | 0 | 1,087 | NEG | NEG |
| H33 | 99,161 | 849,242 | 5,448,051 | POS | POS |

### EXAMPLE 4

In the light of the ability of certain viruses to cause cancer, we tested whether the immunoprecipitation technology employing human papilloma virus (HPV) antigens, could identify blinded sera samples from previously evaluated HPV-DNA positive head and neck tumors. As an initial screening strategy in this pilot study we examined 127 blinded sera samples for high level antibodies against the E7 oncogene of HPV. Sera were evaluated for antibody titers against a Ruc-E7 fusion protein of HPV. High positives were culled, repeated and then this subset was examined for anti-E6 antibodies using a Ruc-E6 fusion protein. Using this less than perfect screening strategy, nine sera were identified by the immunoprecipitation assay as containing high titer antibodies against HPV in this large pilot experiment (Table 6). Following breaking of the blinded code, no HPV-antibody positive calls were made among any of the approximately 60 non-cancer controls. Eight of the nine HPV antibody positives sera were among the head and neck cancer sera samples that were known to have HPV DNA-positive tumors, while three tumors containing high copy number HPV infection were not detected (Table 6). One additionally highly positive HPV immunoreactive sera was also detected, which was not detected as DNA positive and may have represented a head and neck tumor initiated by HPV infection but now resolved or an HPV infection at a different tumor site (e.g. cervical cancer). This pilot study is highly encouraging, in light of additional obvious improvements including using additional HPV gene products to improve sensitivity. It should again be noted that this successful classification was accomplished with crude extracts, was done without optimizing assay conditions and did not involve the use of a training sera set to determine cut-off values. These results with the immunoprecipitation technology profiling exposure to infectious agents should have wide diagnostic applications for HPV-associated cancers and other infection-related cancers.

TABLE 6
Serological Detection of HPV Antibody-Positive Head and Neck
Cancer Sera. 127 blinded sera from non-cancer controls and
head and neck cancer patients, previously analyzed for relative
DNA amounts of HPV DNA in saliva and tumors, were analyzed
for high anti-HPV antibody titers. For determining HPV status,
sera were analyzed for anti-E7 antibody responses, culled
and re-assayed to confirm status.

| | HPV | 10< Copies Tumor |
|---|---|---|
| Head and neck Cancers | Ab Positive | DNA Positive |
| 1203 | − | +++ |
| 1842 | − | +++ |
| 2167 | +++ | − |
| 2405 | +++ | +++ |
| 2674 | +++ | (+) |
| 2704 | +++ | +++ |
| 2715 | +++ | +++ |
| 2723 | +++ | − |
| 2888 | − | +++ |
| 2894 | +++ | +++ |
| 2911 | +++ | +++ |
| 3538 | +++ | +++ |

### REFERENCES

1. Old L J, Chen Y T: New paths in human cancer serology. J Exp Med 1998, 187:1163-1167.
2. Preuss K D, Zwick C, Bormann C, Neumann F, Pfreundschuh M: Analysis of the B-cell repertoire against antigens expressed by human neoplasms. Immunol Rev 2002, 188:43-50.
3. Wadhwa M, Skog A L, Bird C, Ragnhammar P, Lilljefors M, Gaines-Das R, Mellstedt H, Thorpe R: Immunogenicity of granulocyte-macrophage colony-stimulating factor (GM-CSF) products in patients undergoing combination therapy with GM-CSF. Clin Cancer Res 1999, 5:1353-1361.
4. Burbelo P D, Kisailus A E, Peck J W: Detecting protein-protein interactions using Renilla luciferase fusion proteins. Biotechniques 2002, 33:1044-1048, 1050.
5. Vogelstein B, Kinzler K W: Cancer genes and the pathways they control. Nat Med 2004, 10:789-799.
6. Bardelli A, Parsons D W, Silliman N, Ptak J, Szabo S, Saha S, Markowitz S, Willson J K, Parmigiani G, Kinzler K W, Vogelstein B, Velculescu V E: Mutational analysis of the tyrosine kinome in colorectal cancers. Science 2003, 300:949.
7. Wang Z, Shen D, Parsons D W, Bardelli A, Sager J, Szabo S, Ptak J, Silliman N, Peters B A, van der Heijden M S, Parmigiani G, Yan H, Wang T L, Riggins G, Powell S M, Willson J K V, Markowitz S, Kinzler K W, Vogelstein B, Velculescu V E: Mutational analysis of the tyrosine phosphatome in colorectal cancers. Science 2004, 304:1164-1166.
8. Wang Z, Cummins J M, Shen D, Cahill D P, Jallepalli P V, Wang T L, Parsons D W, Traverso G, Awad M, Silliman N, Ptak J, Szabo S, Willson J K V, Markowitz S, Goldberg M L, Karess R, Kinzler K W, Vogelstein B, Velculescu V E, Langauer C: Three classes of genes mutated in colorectal cancers with chromosomal instability. Cancer Res 2004, 64:2998-3001.
9. Rajagopalan H, Jallepalli P V, Rago C, Velculescu V E, Kinzler K W, Vogelstein B, Lengauer C: Inactivation of hCDC4 can cause chromosomal instability. Nature 2004, 428:77-81.
10. Samuels Y, Wang Z, Bardelli A, Silliman N, Ptak J, Szabo S, Yan H, Gazdar A, Powell S M, Riggins G J, Willson J K V, Markowitz S, Kinzler K W, Vogelstein B, Velculescu V E: High frequency of mutations of the PIK3CA gene in human cancers. Science 2004, 304:554.
11. Perou C M, Sorlie T, Eisen M B, van de Rijn M, Jeffrey S S, Rees C A, Pollack J R, Ross D T, Johnsen H, Akslen L A, Fluge O, Pergamenschikov A, Williams C L, Xhu S X, Lonning P E, Borresen-Dale A L, Brown P O, Botstein D: Molecular portraits of human breast tumours. Nature 2000, 406:747-752.
12. van't Veer L J, Dai H, van de Vijver M J, He Y D, Hart A A, Mao M, Peterse H L, van der Kooy K, Marton M J, Witteveen A T, Schreiber G J, Kerkhoven R M, Roberts C, Lisley P S, Bernards R, Friend S H: Gene expression profiling predicts clinical outcome of breast cancer. Nature 2002, 415:530-536.
13. Sorlie T, Perou C M, Tibshirani R, Aas T, Geisler S, Johnsen H, Hastie T, Eisen M B, van de Rijn M, Jeffrey S S, Thorsen T, Quist H, Matese J C, Brown P O, Botstein D, Lonning P E, Borresen-Dale A L: Gene expression patterns of breast carcinomas distinguish tumor subclasses with clinical implications. Proc Natl Acad Sci USA 2001, 98:10869-10874.
14. Sorlie T, Tibshirani R, Parker J, Hastie T, Marron J S, Nobel A, Deng S, Johnsen H, Pesich R, Geisler S, Demeter J, Perou C M, Lonning P E, Brown P O, Borresen-Dale A L, Botstein D: Repeated observation of breast tumor subtypes in independent gene expression data sets. Proc Natl Acad Sci USA 2003, 100:8418-8423.
15. Sotiriou C, Neo S Y, McShane L M, Korn E L, Long P M, Jazaeri A, Martiat P, Fox S B, Harris A L, Liu E T: Breast cancer classification and prognosis based on gene expression profiles from a population-based study. Proc Natl Acad Sci U S A 2003, 100:10393-10398.
16. Takahashi M, Chen W, Byrd D R, Disis M L, Huseby E S, Qin H, McCahill L, Nelson H, Shimada H, Okuno K: Antibody to ras proteins in patients with colon cancer. Clin Cancer Res 1995, 1:1071-1077.
17. Soussi T: p53 Antibodies in the sera of patients with various types of cancer: a review. Cancer Res 2000, 60:1777-1788.
18. Lubin R, Schlichtholz B, Bengoufa D, Zalcman G, Tredaniel J, Hirsch A, de Fromentel C C, Preudhomme C, Fenaux P, Fournier G.: Analysis of p53 antibodies in patients with various cancers define B-cell epitopes of human p53: distribution on primary structure and exposure on protein surface. Cancer Res 1993, 53:5872-5876.
19. Vennegoor C J, Nijman H W, Drijfhout J W, Vernie L, Verstraeten R A, von Mensdorff-Pouilly S, Hilgers J, Verheijen R H, Kast W M, Melief C J, Kenemans P: Autoantibodies to p53 in ovarian cancer patients and healthy women: a comparison between whole p53 protein and 18-mer peptides for screening purposes. Cancer Lett 1997, 116:93-101.
20. Ishikawa T, Fujita T, Suzuki Y, Okabe S, Yuasa Y, Iwai T, Kawakami Y: Tumor-specific immunological recognition of frameshift-mutated peptides in colon cancer with microsatellite instability. Cancer Res 2003, 63:5564-5572.
21. Fensterle J, Becker J C, Potapenko T, Heimbach V, Vetter C S, Brocker E B, Rapp U R: B-Raf specific antibody responses in melanoma patients. BMC Cancer 2004, 4:62.
22. Stockert E, Jager E, Chen Y T, Scanlan M J, Gout I, Karbach J, Arand M, Knuth A, Old L J: A survey of the humoral immune response of cancer patients to a panel of human tumor antigens. J Exp Med 1998, 187:1349-1354.
23. Zhang J Y, Casiano C A, Peng X X, Koziol J A, Chan E K, Tan E M: Enhancement of antibody detection in cancer using panel of recombinant tumor-associated antigens. Cancer Epidemiol Biomarkers Prev 2003, 12:136-143.
24. Bjorck L, Kronvall G: Purification and some properties of streptococcal protein G, a novel IgG-binding reagent. J Immunol 1984, 133:969-974.
25. Utz P J, Gensler T J, Anderson P: Death, autoantigen modifications, and tolerance. Arthritis Res 2000, 2:101-114.
26. Schellekens G A, de Jong B A, van den Hoogen F H, van de Putte L B, van Venrooij W J: Citrulline is an essential constituent of antigenic determinants recognized by rheumatoid arthritis-specific autoantibodies. J Clin Invest 1998, 101:273-281.
27. Yamada R, Suzuki A, Chang X, Yamamoto K: Citrullinated proteins in rheumatoid arthritis. Front Biosci 2005, 10:54-64.
28. Koenen M, Ruther U, Muller-Hill B: Immunoenzymatic detection of expressed gene fragments cloned in the lac Z gene of E. coli. Embo J 1982, 1:509-512.
29. Ruther U, Koenen M, Sippel A E, Muller-Hill B: Exon cloning: immunoenzymatic identification of exons of the chicken lysozyme gene. Proc Natl Acad Sci USA 1982, 79:6852-6855.
30. Phizicky E M, Fields S: Protein-protein interactions: methods for detection and analysis. Microbiol. Rev 1995, 59:94-123.
31. Bode A M, Dong Z: Post-translational modification of p53 in tumorigenesis. Nat Rev Cancer 2004, 4:793-805.
32. Lim Y P: Mining the tumor phosphoproteome for cancer markers. Clin Cancer Res 2005, 11:3163-3169.
33. Hammel P, Boissier B, Chaumette M T, Piedbois P, Rotman N, Kouyoumdjian J C, Lubin R, Delchier J C, Soussi T: Detection and monitoring of serum p53 antibodies in patients with colorectal cancer. Gut 1997, 40:356-361.
34. Crawford L V, Pim D C, Bulbrook R D: Detection of antibodies against the cellular protein p53 in sera from patients with breast cancer. Int J Cancer 1982, 30:403-408.
35. Pirone D M, Fukuhara S, Gutkind J S, Burbelo P D: SPECs, small binding proteins for Cdc42. J Biol Chem 2000, 275:22650-22656.
36. Binley, J. M., P. J. Klasse, Y. Cao, I. Jones, M. Markowitz, D. D. Ho, and J. P. Moore. 1997. Differential regulation of the antibody responses to Gag and Env proteins of human immunodeficiency virus type 1. J. Virol. 71:2799-809.
37. Bishop, L. R., and J. A. Kovacs. 2003. Quantitation of anti-Pneumocystis jiroveci antibodies in healthy persons and immunocompromised patients. J Infect Dis. 187:1844-8.
38. Burbelo, P. D., R. Goldman, and T. L. Mattson. 2005. A simplified immunoprecipitation method for quantitatively measuring antibody responses in clinical sera samples by using mammalian-produced Renilla luciferase-antigen fusion proteins. BMC Biotechnol. 5:22.
39. Daly, K. R., J. Koch, L. Levin, and P. D. Walzer. 2004. Enzyme-linked immunosorbent assay and serologic responses to Pneumocystis jiroveci. Emerg Infect Dis. 10:848-54.
40. Janssen, R. S., G. A. Satten, S. L. Stramer, B. D. Rawal, T. R. O'Brien, B. J. Weiblen, F. M. Hecht, N. Jack, F. R. Cleghorn, J. O. Kahn, M. A. Chesney, and M. P. Busch. 1998. New testing strategy to detect early HIV-1 infection for use in incidence estimates and for clinical and prevention purposes. Jama. 280:42-8.
41. Lin, S., P. Arcangel, A. Medina-Selby, D. Coit, P. Ng, S, Nguyen, C. McCoin, A. Gyenes, C. Hu, L. Tandeske, B. Phelps, and D. Chien. 2005. Design of novel conformational and genotype-specific antigens for improving sensitivity of immunoassays for hepatitis C virus-specific antibodies. J Clin Microbiol. 43:3917-24.
42. Morris, L., J. M. Binley, B. A. Clas, S. Bonhoeffer, T. P. Astill, R. Kost, A. Hurley, Y. Cao, M. Markowitz, D. D. Ho, and J. P. Moore. 1998. HIV-1 antigen-specific and -nonspecific B cell responses are sensitive to combination antiretroviral therapy. J Exp Med. 188:233-45.
43. Neuman de Vegvar, H. E., R. R. Amara, L. Steinman, P. J. Utz, H. L. Robinson, and W. H. Robinson. 2003. Microarray profiling of antibody responses against simian-human immunodeficiency virus: postchallenge convergence of reactivities independent of host histocompatibility type and vaccine regimen. J Virol. 77:11125-38.
44. Schmetzer, O., G. Moldenhauer, R. Riesenberg, J. R. Pires, P. Schlag, and A. Pezzutto. 2005. Quality of recombinant protein determines the amount of autoreactivity detected against the tumor-associated epithelial cell adhesion molecule antigen: low frequency of antibodies against the natural protein. J Immunol. 174:942-52.
45. Schmitt, J. 2003. Recombinant autoantigens for diagnosis and therapy of autoimmune diseases. Biomed Pharmacother. 57:261-8.
46. Vernet, G. 2004. Molecular diagnostics in virology. J Clin Virol. 31:239-47.
47. Zwart, G., L. van der Hoek, M. Valk, M. T. Cornelissen, E. Baan, J. Dekker, M. Koot, C. L. Kuiken, and J. Goudsmit. 1994. Antibody responses to HIV-1 envelope and gag epitopes in HIV-1 seroconverters with rapid versus slow disease progression. Virology. 201:285-93.

1. A method for detecting the presence or concentration of an analyte in a sample, said method comprising:
a) contacting said sample with an immobilized first binding reagent, said reagent capable of binding the analyte if present in the sample;
b) contacting said sample with a second binding reagent which comprises a fusion protein having a reporter domain and a binding domain, said binding domain being capable of binding the analyte if present in the sample, and said first and second binding reagents being capable of binding the analyte simultaneously if present in the sample, such that said second binding reagent becomes immobilized through the analyte bound to the first binding reagent; and
c) detecting whether the second binding reagent has become immobilized to thereby detect the presence or concentration of said analyte.

2. The method of claim 1, wherein the analyte is selected from the group consisting of a protein, and antibody, a carbohydrate and a lipid.

3. The method of claim 1, wherein the analyte is an antibody.

4. The method of claim 1, wherein the analyte is a protein.

5. The method of claim 3, wherein the antibody is an IgA, an IgE, an IgG or an IgM.

6. The method of claim 3, wherein the first binding reagent is a protein.

7. The method of claim 1, wherein the reporter domain of the second binding reagent comprises a detectable enzyme.

8. The method of claim 1, wherein the reporter domain of the second binding reagent comprises multiple copies of a detectable enzyme.

9. The method of claim 7, wherein the detectable enzyme is selected from the group consisting of luciferase, horseradish peroxidase, and alkaline phosphatase.

10. The method of claim 9, wherein the detectable enzyme comprises luciferase.

11. The method of claim 1, wherein the binding domain of said second binding reagent comprises at least the antigenic portion of a tumor-associated protein.

12. The method of claim 11, wherein multiple second binding reagents are used, each one comprising a different portion of a different tumor-associated protein.

13. The method of claim 11, wherein the binding domain of said second binding reagent comprises at least the antigenic portion of a protein selected from the group consisting of p53, K-Ras, c-Myc, β-catenin and Smad4, and mixtures thereof.

14. The method of claim 1, wherein the fusion protein is expressed in a mammalian cell.

15. The method of claim 1, wherein the sample comprises a body fluid.

16. The method of claim 15, wherein the body fluid is selected from the group consisting of blood, saliva, ascites, urine, cerebrospinal fluid, sputum, amniotic fluid and gastric fluid.

17. The method of claim 6, wherein the first binding reagent comprises immobilized protein A.

18. The method of claim 6, wherein the first binding reagent comprises immobilized protein G.

19. The method of claim 11, wherein the presence of the analyte is indicative of the presence of a tumor in the organism from which the sample originated.

20. The method of claim 19, wherein the tumor is selected from the group consisting of colon cancer, breast cancer, prostate cancer, and head and neck cancer.

21. The method of claim 1, wherein the presence of the analyte is indicative of the presence of an infectious agent in the organism from which the sample originated.

22. The method of claim 21, wherein the infectious agent comprises a virus, a bacterium, a fungus or a parasite.

23. The method of claim 22, wherein the virus is HIV, CMV, HPV, RSV, Hepatitis B, Hapatitis C, West Nile, herpes, SARS, or HTLV-1.

24. The method of claim 1, wherein the presence of the analyte is indicative of the presence of an allergic reaction.

25. The method of claim 1, wherein step a) is carried out prior to step b).

26. The method of claim 1, wherein step b) is carried out prior to step a).

27. The method of claim 1, wherein step a) and step b) are carried out simultaneously.

28. The method of claim 1, wherein multiple second binding reagents are used.

29. The method of claim 28, wherein the multiple second binding reagents bind to different sites on the analyte.

30. The method of claim 28, wherein each multiple second binding reagent is capable of binding to a different analyte.

31. The method of claim 14, wherein the second binding reagent includes a post-translational modification.

32. The method of claim 1, wherein the binding domain of the second binding reagent comprises a full-length protein.

33. The method of claim 1, wherein the binding domain of the second binding reagent comprises a portion of a full-length protein sufficient to bind to the analyte.

34. The method of claim 14, wherein the mammalian cell is a human cell.

35. The method of claim 1, wherein the second binding reagent is a component of a crude extract.

36. A method for monitoring the course of a disease in a patient having need of such monitoring, said method comprising:
a) contacting a first fluid sample from said patient with an immobilized first binding reagent, said reagent capable of binding to an analyte in the sample whose level is indicative of the state of the disease;
b) contacting said sample with a second binding reagent which comprises a fusion protein having a reporter domain and a binding domain, said binding domain being capable of binding the analyte if present in the sample, and said first and second binding reagents being capable of binding the analyte simultaneously, such that said second binding reagent becomes immobilized through the first binding reagent;
c) detecting the extent to which the second binding reagent has become immobilized to thereby quantify the concentration of said analyte; and
d) repeating steps (a)-(c) on a second fluid sample collected from said patient at a time subsequent to the collection of the first fluid sample to thereby monitor the concentration of the analyte over time.

37. The method of claim 36, wherein the analyte is selected from the group consisting of a protein, an antibody, a carbohydrate and a lipid.

38. The method of claim 36, wherein the analyte is an antibody.

39. The method of claim 36, wherein the analyte is a protein.

40. The method of claim 38, wherein the antibody is an IgA, an IgE, an IgG or an IgM.

41. The method of claim 38, wherein the first binding reagent is a protein.

42. The method of claim 36, wherein the reporter domain of the second binding reagent comprises a detectable enzyme.

43. The method of claim 36, wherein the reporter domain of the second binding reagent comprises multiple copies of a detectable enzyme.

44. The method of claim 42, wherein the detectable enzyme is selected from the group consisting of luciferase, horseradish peroxidase, and alkaline phosphatase.

45. The method of claim 44, wherein the detectable enzyme comprises luciferase.

46. The method of claim 36, wherein the binding domain of said second binding reagent comprises at least the antigenic portion of a tumor-associated protein.

47. The method of claim 46, wherein multiple second binding reagents are used, each one comprising a different portion of a different tumor-associated protein.

48. The method of claim 46, wherein the binding domain of said second binding reagent comprises at least the antigenic portion of a protein selected from the group consisting of p53, K-Ras, c-Myc, β-catenin and Smad4, and mixtures thereof.

49. The method of claim 36, wherein the fusion protein is expressed in a mammalian cell.

50. The method of claim 36, wherein the samples comprise a body fluid.

51. The method of claim 50, wherein the body fluid is selected from the group consisting of blood, saliva, ascites, urine, cerebrospinal fluid, sputum and gastric fluid.

52. The method of claim 41, wherein the first binding reagent comprises immobilized protein A.

53. The method of claim 41, wherein the first binding reagent comprises immobilized protein G.

54. The method of claim 46, wherein the presence of the analyte is indicative of the presence of a tumor in the organism from which the sample originated.

55. The method of claim 54, wherein the tumor is selected from the group consisting of colon cancer, breast cancer, prostate cancer, and head and neck cancer.

56. The method of claim 36, wherein the presence of the analyte is indicative of the presence of an infectious agent in the organism from which the sample originated.

57. The method of claim 56, wherein the infectious agent is selected from a virus, a bacterium, a fungus and a parasite.

58. The method of claim 57, wherein the virus is HIV, CMV, HPV, RSV, Hepatitis B, Hapatitis C, West Nile, herpes, SARS, or HTLV-1.

59. The method of claim 36, wherein the presence of the analyte is indicative of the presence of an allergic reaction.

60. The method of claim 36, wherein step a) is carried out prior to step b).

61. The method of claim 36, wherein step b) is carried out prior to step a).

62. The method of claim 36, wherein step a) and step b) are carried out simultaneously.

63. The method of claim 36, wherein multiple second binding reagents are used when testing a fluid sample.

64. The method of claim 63, wherein the multiple second binding reagents bind to different sites on the analyte.

65. The method of claim 63, wherein each multiple second binding reagent is capable of binding to a different analyte.

66. The method of claim 49, wherein the second binding reagent includes a post-translational modification.

67. The method of claim 36, wherein the binding domain of the second binding reagent comprises a full-length protein.

68. The method of claim 36, wherein the binding domain of the second binding reagent comprises a portion of a full-length protein sufficient to bind to the analyte.

69. The method of claim 49, wherein the mammalian cell is a human cell.

70. The method of claim 36, wherein the second binding reagent is a component of a crude extract.

71. A kit for detecting the presence or concentration of an analyte in a sample, said kit comprising:
d) an immobilized first binding reagent, said reagent capable of binding the analyte if present in the sample;
e) a second binding reagent which comprises a fusion protein having a reporter domain and a binding domain, said binding domain being capable of binding the analyte if present in the sample, and said first and second binding reagents being capable of binding the analyte simultaneously if present in the sample, such that said second binding reagent becomes immobilized through the first binding reagent; and
f) suitable packaging material.

72. The kit of claim 71, which further comprises instructional material for use of said kit.

73. The kit of claim 71, which further comprises a positive control.

74. The kit of claim 71, which further comprises a reagent for detecting the reporter domain of the second binding reagent.

75. A method for detecting the presence or concentration of an analyte in a sample, said method comprising, in any order:
d) contacting said sample with a first binding reagent, said reagent capable of binding the analyte if present in the sample;
e) contacting said sample with a second binding reagent which comprises a fusion protein having a reporter domain and a binding domain, said binding domain being capable of binding the analyte if present in the sample, and said first and second binding reagents being capable of binding the analyte simultaneously if present in the sample;
f) immobilizing said first binding reagent such that when the first and second binding reagents are bound to the analyte, said second binding reagent becomes immobilized through the analyte bound to the first binding reagent; and
g) detecting whether the second binding reagent has become immobilized to thereby detect the presence or concentration of said analyte.
